(19) United States
(12) Patent Application Publication
(10) Pub. No.: US 2022/0139541 A1
(43) Pub. Date: May 5, 2022

(54) PART REPLACEMENT REGISTRATION TOOL

(71) Applicant: KONINKLIJKE PHILIPS N.V., EINDHOVEN (NL)

(72) Inventors: Johannes Henricus Maria KORST, EINDHOVEN (NL); Mauro BARBIERI, EINDHOVEN (NL); Serverius Petrus Paulus PRONK, VUGHT (NL); Marc André PETERS, VELDHOVEN (NL)

(21) Appl. No.: 17/515,904

(22) Filed: Nov. 1, 2021

Related U.S. Application Data
(60) Provisional application No. 63/108,907, filed on Nov. 3, 2020.

(51) Int. Cl. G16H 40/40; G16H 40/60; G06Q 10/00

(52) U.S. Cl. CPC G16H 40/40; G06Q 10/20; G16H 40/60

(57) ABSTRACT

An apparatus (100) includes at least one electronic processor (101, 113) programmed to: provide a parts ordering system (122) for ordering replacement parts for medical devices, the parts ordering system utilizing the display device and the at least one user input device and identifying replacement parts by part-type-number (PTN) (134); maintain a database (130) of possible extracted PTNs (136) corresponding to PTNs of replacement parts available for order via the parts ordering system; generate a list (140) of possible extracted parts, from data stored in the database, including at least one possible extracted PTN corresponding to a PTN of a replacement part ordered via the parts ordering system; identify a PTN of an extracted part from the list of possible extracted parts; and store, in a database (138) of part replacement events, the PTN of the replacement part and the PTN of the extracted part.

### CROSS REFERENCE TO RELATED APPLICATIONS

This application claims the benefit of U.S. Provisional Patent Application No. 63/108,907 filed Nov. 3, 2020. This application is hereby incorporated by reference herein.

### FIELD

The following relates generally to the medical device maintenance arts, part replacement registration arts, replacement part identification tracking arts, and related arts.

### BACKGROUND

When a service technician (remote or on-site) or customer service representative or the like assists a customer (for example, with a medical imaging device having some type of problem), a parts ordering system is typically used to order replacement parts as needed for the servicing or repair task. The parts ordering system is usually an electronic application, for example implemented as a backend server computer that is accessible via an application program running on a desktop or laptop computer, and/or via an application program (app) running on a cellphone, tablet, or other mobile device. Usually, the user of the parts ordering system can identify the replacement part to be ordered by entering the name (or partial name) of the part, and the parts ordering system performs a search on a database of replacement parts available for order via the parts ordering system. The search returns the part type number (PTN) or PTNs corresponding to the (possibly partial) name of the replacement part. The user selects the PTN to be ordered and may provide other information (shipping address information et cetera) to complete the order. If the user happens to know the PTN at the time of ordering, the user may be able to directly enter the PTN thereby bypassing the name→PTN search phase. It should be noted that the PTN may be identified by some other phraseology, such as a part number—the term “part type number” or PTN is intended to encompass these various alternative phraseologies. Furthermore, the PTN is not limited to numerical quantities, but rather may be made up of numerals, alphabetic letters, and/or possibly other symbols such as dashes (“-”), periods (“.”), or so forth. Part type numbers are different than serial numbers that should be unique for each individual part. Serial numbers are not used for many part types.

For the maintenance of complex equipment such as, e.g., a medical system, there is a growing tendency to use preventive maintenance, where the condition of the medical system is continuously monitored so that some of the parts can be extracted preventively before they actual fail. In the case of complex and computerized equipment such as a medical imaging device, it is often the case that the medical imaging device automatically maintains a log of events (for example, information on imaging sessions, configuration settings/setting changes, sensor readings, warnings or alerts generated by components of the medical imaging device, and/or so forth). Data collected in this “machine” log may be occasionally transmitted (e.g., on a daily schedule) to a service center run by the medical imaging device manufacturer or other entity that provides servicing for the medical imaging device. Using log data and part replacement data of historical maintenance cases, failure prediction models are applied against the log and part replacement data at the service center. These models have been developed to predict when a specific part is likely to fail. For example, the failure prediction model may employ artificial intelligence (AI) such as an artificial neural network (ANN), support vector machine (SVM) model, or the like that has been trained on past historical machine log data annotated with actual part failures. The model may, for example, output a probability of failure in a specified time frame. In addition, parts replacement data is used to investigate the reliability of individual parts, preferably on the level of individual part type numbers.

The following discloses certain improvements to these processes.

### SUMMARY

In one aspect, an apparatus for servicing one or more medical devices includes a display device; at least one user input device; and at least one electronic processor programmed to: provide a parts ordering system for ordering replacement parts for medical devices, the parts ordering system utilizing the display device and the at least one user input device and identifying replacement parts by PTN; maintain a database of possible extracted PTNs corresponding to PTNs of replacement parts available for order via the parts ordering system; generate a list of possible extracted parts, from data stored in the database, including at least one possible extracted PTN corresponding to a PTN of a replacement part ordered via the parts ordering system; identify a PTN of an extracted part from the list of possible extracted parts; and store, in a database of part replacement events, the PTN of the replacement part and the PTN of the extracted part.

In another aspect, a non-transitory computer readable medium stores instructions executable by at least one electronic processor to perform a method of servicing a medical device. The method includes: providing a parts ordering system for ordering replacement parts for medical devices, the parts ordering system utilizing the display device and the at least one user input device and identifying replacement parts by PTN; maintaining a database of possible extracted PTNs corresponding to PTNs of replacement parts available for order via the parts ordering system; generating a list of possible extracted parts, from data stored in the database, including at least one possible extracted PTN corresponding to a PTN of a replacement part ordered via the parts ordering system; displaying the list of possible extracted parts on a display device; receiving entry of the PTN of the extracted part from the user via at least one user input device; identifying the entered PTN of an extracted part from the list of possible extracted parts; and storing, in a database of part replacement events, the PTN of the replacement part and the PTN of the extracted part.

In another aspect, a service device includes a display device; at least one user input device; and at least one electronic processor programmed to: display, on the display device, a parts ordering system for ordering replacement parts for medical devices, the parts ordering system utilizing the display device and the at least one user input device and identifying replacement parts by PTN; display, on the display device, a list of possible extracted parts, from data stored in a database, including at least one possible extracted PTN corresponding to a PTN of a replacement part ordered via the parts ordering system; receive entry of the PTN of the extracted part from the user via the at least one user input device; identify the entered PTN of an extracted part from the list of possible extracted parts; and display, on the display device, a visualization of a replacement part from the identified extracted part.

One advantage resides in providing an efficient tool for receiving information on extracted parts during the ordering replacement parts for a medical device or other complex equipment undergoing service.

Another advantage resides in providing a parts ordering system with a tool for receiving information on extracted parts during the ordering of replacement parts for a medical device or other complex equipment undergoing service.

Another advantage resides in providing updated identification numbers for replacement parts for a medical device undergoing service.

Another advantage resides in increasing providing for automated or semi-automated collection of parts replacement data including both replacement parts and corresponding extracted parts for use in analysis tasks such as training failure prediction models and investigating reliability of individual parts.

Another advantage resides in reconciling multiple identification numbers for replacement parts for a medical device undergoing service.

Another advantage resides in providing entry of identification of an extracted part for ordering a replacement part for a medical device undergoing service.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for supporting a service engineer in servicing a device in accordance with the present disclosure.

FIG. 2 shows exemplary flow chart operations of the system of FIG. 1.

### DETAILED DESCRIPTION

For both failure prediction and reliability analysis it is important to have accurate information of part replacements for historical maintenance cases. To do these analyses with sufficient accuracy, it is important to have the part type numbers of the parts concerned.

Part replacement data of historical maintenance cases obtained from parts orders placed using a parts ordering system contain information on the new parts that have been ordered and are placed into the medical system to resolve the issue, i.e., information on the so-called replacement parts. However, the replacement part ordering process conventionally does not provide additional information on the parts that have been taken out of the system to resolve the issue, i.e., information on the so-called extracted parts. More specifically, there is little or no information on the part type numbers (PTNs) of the extracted parts. Field service engineers (FSEs) working to resolve a failure of a given medical system often work under a high time pressure, as the hospital wants to start using the system as soon as possible. In these circumstances, accurately keeping track of the part type numbers of the extracted parts is considered of low priority. A similar situation can arise in field servicing of other complex equipment such as commercial airliners (where an airliner undergoing repair represents lost passenger and/or cargo shipping revenue), industrial manufacturing equipment (where a machine undergoing repair can sometimes shut down an entire assembly or processing line), and so forth.

The following relates to a current service call application (“app”) used by a FSE performing maintenance on a medical imaging device that provides for entry of a replacement part (that is, a part that is being installed in the imaging device as a replacement). In some embodiments, the service call app may interface the FSE with a parts ordering system. In some embodiments, the service call app may be a reporting app via which the FSE prepares and files a report on the service call. In some embodiments, the service call app may provide both interfacing with a parts ordering system and also provide for service call reporting. The service call app may optionally also include other functionality useful to the FSE, such as providing a calls schedule, providing secure communication with a supervisor, and/or so forth. To assist the FSE in selecting the replacement part, the app provides a replace-by table that indicates, for each part, whether newer versions (or makes, or models) of the part are backward compatible (that is, the older version can be extracted by the newer version but not vice versa) or fully compatible (that is, the older version can be extracted by the newer version and the newer version can be extracted by the older version).

The replace-by table can include an “old” part type number (PTN) and “new” PTN pair. Advantageously, embodiments disclosed herein provide a mechanism for providing entry of the extracted part, that is, the part that is being extracted by the replacement part, in addition to entering the replacement part to be (or which has been) ordered and/or installed in the medical device.

Initially, it might seem that the extracted part should be identical to the replacement part. However, this is often not the case because there may be multiple versions, makes, or models of a given part due to ongoing updates of the part by a given manufacturer or due to use of third-party parts from different suppliers.

Even so, it might seem that the extracted part could be determined from the service log of the imaging device, as the extracted part in the current service call would be the replacement part of a prior service call. However, this also is not always the case. For example, there may be multiple instances of a given part in the imaging device, e.g. multiple displays of the same type, multiple cables of the same type, or so forth. In such cases, neither the identity of the extracted part nor its location is known based on the service log as presently constituted.

Hence, the following discloses an improvement in which the service call app is modified to provide for entry of a part type number (PTN) identifying the extracted part. In one approach, the replace-by table is referenced to identify all possible extracted parts corresponding to the replacement part, and these are listed in a drop-down list or other GUI dialog so that the FSE merely needs to select the PTN of the extracted part. The app preferably asks for entry of the PTN of the extracted part at the time, or sometime after, the FSE provides the log entry recording the replacement part.

Additionally, if the imaging device has multiple instances of the part, then the app provides for also entering the location of the extracted part. This can be done in various ways, such as describing the location in relative terms (e.g., extracted display is the left-side display”) or, for cables, by specifying the parts connected by the cables, or for some other parts a graphical representation may be shown to identify the various locations, and the FSE enters the location by clicking on the correct location in the graphical representation.

In some embodiments disclosed herein, the automatically generated log file of the imaging device is accessed to detect changes in serial numbers of parts, and from this both the replacement part and the extracted part can be automatically identified. However, this is only an option for larger components, as the serial numbers of cables and other consumables or small items are not typically logged.

In other embodiments disclosed herein, the provided information including both the replacement part and the extracted part can be leveraged to provide various warnings or notifications. For example, if the replacement part is a newer version versus the extracted part and this version update also requires a software upgrade and/or upgrading some other parts of the imaging device, a notification to this effect can be provided.

The information collected including both the replacement part and the extracted part can be leveraged to perform various analyses, such as part reliability analysis or failure prediction.

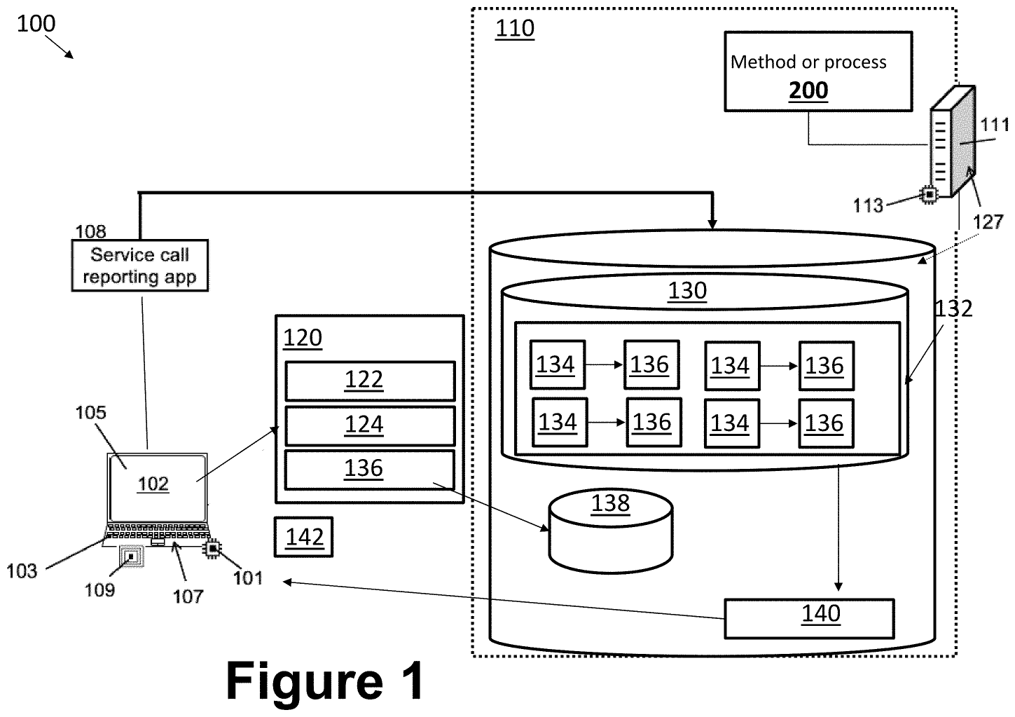

With reference to FIG. 1, an illustrative servicing support system 100 for supporting a service engineer in servicing a device (e.g., a patient monitor or a medical imaging device, not shown—also referred to as a medical device, an imaging device, imaging scanner, and variants thereof) is diagrammatically shown. By way of some non-limiting illustrative examples, the medical imaging device under service may be a magnetic resonance imaging (MM) scanner, a computed tomography (CT) scanner, a positron emission tomography (PET) scanner, a gamma camera for performing single photon emission computed tomography (SPECT), an interventional radiology (IR) device, or so forth. As shown in FIG. 1, the servicing support system 100 includes, or is accessible by, a service device 102 carried or accessed by a FSE. The service device 102 can be a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g. notebook computer, tablet computer, or so forth) carried by a FSE performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer and the servicing support system 100 may be embodied as an “app” (application program). The service device 102 allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 to implement the servicing support system 100. The service device 102 may also include a communication interface 109 such that the servicing support system 100 may communicate with a backend server or processing device 111, which may optionally implement some aspects of the servicing support system 100 (e.g., the server 111 may have greater processing power and therefore be preferable for implementing computationally complex aspects of the servicing support system 100). Such communication interfaces 109 include, for example, a wireless Wi-Fi or 4G/5G interface, a wired Ethernet interface, or the like for connection to the Internet and/or an intranet. Some aspects of the servicing support system 100 may also be implemented by cloud processing or other remote processing.

In illustrative FIG. 1, the servicing information collected using a service call reporting app 108 is fed to a database backend 110 (e.g., implemented at a medical facility or other remote center from where the FSE is performing the service call, or at the imaging device vendor or other servicing contractor). For example, the database backend 110 may implement a service log for the medical imaging device. The backend processing is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single service device 102, more generally the database backend 110 will receive service call reports from many service devices (e.g., tens, hundreds, or more service devices) carried by different FSEs, and each FSE will be providing a service call report for each service call that the FSE makes (this may total hundreds or even a few thousand service calls per year by a given FSE). Hence, over time the database backend 110 accumulates a large quantity of service call reporting data.

The display device 105 is configured to display a graphical user interface (GUI) 120 that includes a GUI dialog of a parts ordering system 122 for ordering replacement parts for medical devices. The GUI 120 also includes an entry GUI dialog 124 for entry of a location of the extracted part of one of the medical devices.

The non-transitory computer readable medium 127 stores a part-type-numbers (PTNs) database 130 of PTNs corresponding to PTNs of replacement parts available for order via the parts ordering system 122. In some embodiments, the PTN database 130 comprises a replace-by table 132 storing pairs of PTNs 134 of replacement parts and PTNs 136 of possible extracted parts (that is, parts that could be extracted by the given replacement part). The non-transitory computer readable medium 127 also stores a part replacement events database 138 of replacement parts numbers. Although shown in FIG. 1 as separate databases, in some examples, the PTN database 130 and the part replacement events database 138 can comprise a single database.

The non-transitory storage medium 127 stores instructions executable by the electronic processor 113 of the backend server 111 to perform a method 200 of servicing one or more medical devices.

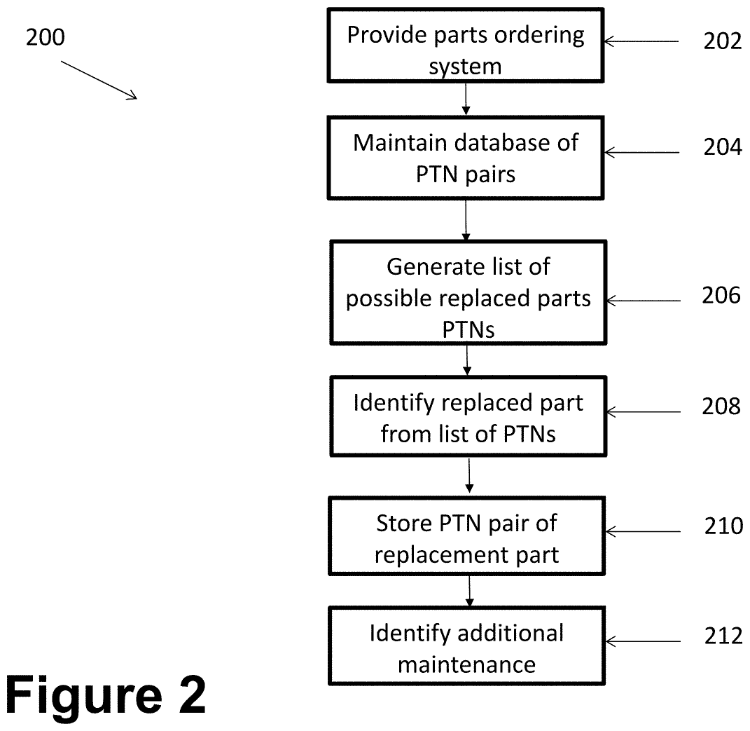

With reference to FIG. 2, and with continuing reference to FIG. 1, an illustrative embodiment of an instance of the device service support method 200 executable by the electronic processor 113 is diagrammatically shown as a flowchart. In some examples, the method 200 may be performed at least in part by cloud processing.

At an operation 202, a FSE provides an input, via the at least one user input device 103, to the service device 102 to bring up the parts ordering system 122 on the GUI 120. The electronic processor 113 of the backend server 111 is programmed to provide the parts ordering system 122 on the GUI 120. The parts ordering system 122 utilizes the display device 105 and the at least one user input device 103. The parts ordering system 122 can then be used by the FSE to identify (and optionally actually order) replacement parts for the medical device by PTNs 134, 136 retrieved from the PTN database 130. In another approach for identifying the replacement part, the FSE may enter the name of the part, or an initial portion of the name of the part (e.g., “USB cable” or “USB cab”) and the backend server 111 queries a parts name/PTN database (not shown) to identify all PTNs 134, 136 associated to that (possibly partial) name. The PTNs 134, 136 returned by the query are presented to the FSE, e.g. as a list, and optionally including images of the parts corresponding to the PTNs, and the FSE selects the PTN to be ordered from the list. Other approaches are also contemplated, such entering the (possibly partial) name and the make/model of the imaging device under repair to bring up the PTNs 134, 136 that match the name and also are compatible with that make/model of imaging device. The parts ordering system 122 optionally provides additional functionality such as actually placing an order for the entered or selected replacement part including providing for entry of a delivery address, receiving and processing customer billing information, and/or so forth.

At an operation 204, the PTN database 130 is maintained to include possible extracted PTNs 136 corresponding to PTNs of replacement parts 134 available for order via the parts ordering system 122. The operation 204 can typically be continuously performed before the parts ordering system 122 is provided to the service device 102.

At an operation 206, a list 140 of possible extracted parts is generated from data stored in the PTN database 130. The data can include, for example, one or more possible extracted PTNs 136 that correspond to a PTN 134 of a replacement part ordered via the parts ordering system 122 by the FSE. In performing the operation 206, other relevant information may optionally also be leveraged to reduce the length of the list 140 of possible extracted PTNs 136. For example, if the make/model of the imaging device undergoing repair is provided (e.g. entered during the parts ordering process) then the list 140 of possible extracted PTNs 136 may be filtered to remove any PTNs that are not compatible with that make/model of imaging device.

At an operation 208, a PTN 136 of an extracted part is identified from the list of possible extracted parts. In some embodiments, the identification operation 208 includes querying the list 140 of possible extracted parts against a log file of a medical device associated with the replacement part ordered via the parts ordering system 122 to automatically identify the extracted part. This includes the FSE using the parts ordering system 122 to identify the medical device being serviced, so that the medical device is associated with the replacement part. The query would look for an instance of one of the possible extracted parts in the log file to identify the actual extracted part.

In other embodiments, the identification operation 208 includes displaying the list 140 of possible extracted parts on the display device 105. The FSE then selects, via the at least one user input device 103, an entry of the PTN 136 of the extracted part from the user. The list 140 can then be queried to find the entered PTN 136 to identify the extracted part corresponding to the PTN 134 of the replacement part. In some examples, the FSE can be assisted in easily finding the correct extracted part. To do so, visuals of the possible alternative extracted parts can be displayed on the display device 105. These visuals emphasize the difference between these alternatives, for example by providing additional text explaining the differences, or by encircling on the visuals the details on which the FSEs should focus their attention to quickly identify the differences.

The above-two embodiments are not necessarily mutually exclusive. For example, in a combined approach the log file of the medical device is first queried to attempt to automatically identify the extracted part, and if this fails or returns an ambiguous result then the second approach of displaying the list 140.

A possible issue can arise if there are multiple instances of the part in question in the medical device. For example, if the replacement part is a USB cable, there may be two, or more, or many USB cables in a medical device. Hence, for failure analysis and some other tasks it may be useful to know which of these multiple instances is undergoing replacement. To address this, the entry GUI dialog 124 for entry of a location of the extracted part of one of the medical devices is displayed on the display device 105. The entry GUI dialog 124 includes a graphical representation on the GUI 120 showing one or more possible locations of the extracted part. The entry of the location includes receiving an indication of one of the possible locations of the extracted part via the at least one user input device 103.

At an operation 210, the PTN of the replacement part and the PTN of the extracted part (and optionally also its location, in the case of a replacement part for which there are multiple instances) are stored in the part replacement events database 138. The data in the part replacement events database 138 can be used in future service visits by the FSE. In some examples, the entered location of the extracted part is stored in the part replacement events database 136 along with the PTN of the replacement part and the PTN of the extracted part.

At an operation 212, additional maintenance or parts replacement can be identified based on the PTN 134 of the replacement part and the PTN 136 of the extracted part. The additional maintenance might, for example, include performing a software upgrade to implement new features supported by the replacement part that were not available with the extracted part. An example of an additional parts replacement might be if the replacement part requires a different kind of fastener or securing mechanism. In some embodiments, further maintenance of the medical device can include identifying further required maintenance and/or parts replacement based on the PTN of the extracted part 136 and the PTN of the replacement part 134, and outputting a notification (e.g., a visual message on the display device 105, an audio notification out via a loudspeaker (not shown), and so forth) of the identified further maintenance. In another embedment, a required additional replacement part can be identified based on the PTN of the extracted part 136 and the PTN 134 of the replacement part and outputting a notification the required additional replacement part. In some examples, the query of the list 140 can include detecting one or more changes in serial numbers of one or more previously-extracted parts and identifying the extracted part from the detected changes in the serial numbers. A visualization, such as the PTN 134, of the replacement part can be displayed on the display device 105.

In some embodiments, a replacement part takes over the functionality of multiple extracted parts. Due to further integration of components, a subsystem that was previously implemented with multiple parts can be extracted by a single replacement part. In such cases, the replacement history of a system is not registered as a sequence of pairs of parts, but as a sequence of pairs of sets of parts ((S1, S′1), (S2, S′2), . . . ), where most often S1 and S′1 each contain only one part (but occasionally S1 contains multiple parts, while S′1 contains only one part. It is even possible that S1 contains only one part, while S′1 contains two parts. For example, if the single part in S1 is a component of which a subpart wears out more quickly than the rest of the component. The design could then be adapted so that only the subpart can be extracted. The first time that a replacement of the given component is needed, it would be extracted by two parts, of which one is the subpart. In subsequent cases, only this subpart would have to be extracted.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus for servicing one or more medical devices, the apparatus comprising:
a display device;
at least one user input device; and
at least one electronic processor programmed to:
provide a parts ordering system for ordering replacement parts for medical devices, the parts ordering system utilizing the display device and the at least one user input device and identifying replacement parts by part-type-number (PTN);
maintain a database of possible extracted PTNs corresponding to PTNs of replacement parts available for order via the parts ordering system;
generate a list of possible extracted parts, from data stored in the database, including at least one possible extracted PTN corresponding to a PTN of a replacement part ordered via the parts ordering system;
identify a PTN of an extracted part from the list of possible extracted parts; and
store, in a database of part replacement events, the PTN of the replacement part and the PTN of the extracted part.

2. The apparatus of claim 1, wherein the identification of the PTN of the extracted part includes:
displaying the list of possible extracted parts on the display device; and
receiving entry of the PTN of the extracted part from the user via at least one user input device.

3. The apparatus of claim 1, wherein the identification of the PTN of the extracted part includes:
querying the list of possible extracted parts against a log file of a medical device associated with the replacement part ordered via the parts ordering system to identify the extracted part.

4. The apparatus of claim 3, wherein the querying includes:
detecting one or more changes in serial numbers of one or more previously-extracted parts;
identifying the extracted part from the detected changes in the serial numbers.

5. The apparatus of claim 1, wherein the database comprises a replace-by table storing pairs of PTNs of replacement and extracted parts.

6. The apparatus of claim 2, wherein the identification of the PTN of the extracted part further includes:
displaying, on the display device, a GUI dialog for entry of a location of the extracted part of one of the medical devices;
wherein the entered location of the extracted part is stored in the database of part replacement events along with the PTN of the replacement part and the PTN of the extracted part.

7. The apparatus of claim 6, wherein the GUI dialog for entry of the location of the extracted part includes a graphical representation on a GUI showing one or more possible locations of the extracted part, and the entry of the location includes receiving an indication of one of the possible locations of the extracted part via the at least one user input device.

8. The apparatus of claim 1, wherein the at least one electronic processor is further programmed to:
identify required further maintenance based on the PTN of the extracted part and the PTN of the replacement part; and
output a notification of the identified further maintenance.

9. The apparatus of claim 1, wherein the at least one electronic processor is programmed to:
identify a required additional replacement part based on the PTN of the extracted part and the PTN of the replacement part; and
output a notification of the required additional replacement part.

10. The apparatus of claim 1, wherein the medical device comprises a medical imaging device or a patient monitor.

11. A non-transitory computer readable medium storing instructions executable by at least one electronic processor to perform a method of servicing a medical device, the method including:
providing a parts ordering system for ordering replacement parts for medical devices, the parts ordering system utilizing the display device and the at least one user input device and identifying replacement parts by part-type-number (PTN);
maintaining a database of possible extracted PTNs corresponding to PTNs of replacement parts available for order via the parts ordering system;
generating a list of possible extracted parts, from data stored in the database, including at least one possible extracted PTN corresponding to a PTN of a replacement part ordered via the parts ordering system;
displaying the list of possible extracted parts on a display device;
receiving entry of the PTN of the extracted part from the user via at least one user input device;
identifying the entered PTN of an extracted part from the list of possible extracted parts; and
storing, in a database of part replacement events, the PTN of the replacement part and the PTN of the extracted part.

12. The non-transitory computer readable medium of claim 11, wherein the database comprises a replace-by table storing pairs of PTNs of replacement and extracted parts.

13. The non-transitory computer readable medium of claim 11, wherein the identification of the PTN of the extracted part further includes:
displaying, on the display device, a GUI dialog for entry of a location of the extracted part of one of the medical devices;
wherein the entered location of the extracted part is stored in the database of part replacement events along with the PTN of the replacement part and the PTN of the extracted part.

14. The non-transitory computer readable medium of claim 13, wherein the GUI dialog for entry of the location of the extracted part includes a graphical representation on a GUI showing one or more possible locations of the extracted part, and the entry of the location includes receiving an indication of one of the possible locations of the extracted part via the at least one user input device.

15. The non-transitory computer readable medium of claim 14, wherein the identification of the PTN of the extracted part further includes:
detecting one or more changes in serial numbers of one or more previously-extracted parts;
identifying the extracted part from the detected changes in the serial numbers.

16. The non-transitory computer readable medium of claim 11, wherein the method is further programmed to:
identifying required further maintenance based on the PTN of the extracted part and the PTN of the replacement part; and
outputting a notification of the identified further maintenance.

17. The non-transitory computer readable medium of claim 11, wherein the method is further programmed to:
identifying a required additional replacement part based on the PTN of the extracted part and the PTN of the replacement part; and
outputting a notification of the required additional replacement part.

18. A service device, comprising:
a display device;
at least one user input device; and
at least one electronic processor programmed to:
display, on the display device, a parts ordering system for ordering replacement parts for medical devices, the parts ordering system utilizing the display device and the at least one user input device and identifying replacement parts by part-type-number (PTN);
display, on the display device, a list of possible extracted parts, from data stored in a database, including at least one possible extracted PTN corresponding to a PTN of a replacement part ordered via the parts ordering system;
receive entry of the PTN of the extracted part from the user via the at least one user input device; and
identify the entered PTN of an extracted part from the list of possible extracted parts; and
display, on the display device, a visualization of a replacement part from the identified extracted part.

19. The service device of claim 18, wherein the identification of the PTN of the extracted part further includes:
displaying, on the display device, a GUI dialog for entry of a location of the extracted part of one of the medical devices;
wherein the GUI dialog for entry of the location of the extracted part includes a graphical representation on a GUI showing one or more possible locations of the extracted part, and the entry of the location includes receiving an indication of one of the possible locations of the extracted part via the at least one user input device.

20. The service device of claim 18, wherein the database comprises a replace-by table storing pairs of PTNs of replacement and extracted parts.
